(19) United States
(12) Patent Application Publication
(10) Pub. No.: US 2014/0088407 A1
(43) Pub. Date: Mar. 27, 2014

(54) Magnetic Resonance Angiography using Velocity-Selective Magnetization Preparation

(71) Applicants: Taehoon Shin, Seoul (KR); Bob S. Hu, Los Altos, CA (US); Dwright G. Nishimura, Palo Alto, CA (US)

(72) Inventors: Taehoon Shin, Seoul (KR); Bob S. Hu, Los Altos, CA (US); Dwright G. Nishimura, Palo Alto, CA (US)

(21) Appl. No.: 14/034,798

(22) Filed: Sep. 24, 2013

Related U.S. Application Data
(60) Provisional application No. 61/704,619, filed on Sep. 24, 2012.

(51) Int. Cl. A61B 5/055; A61B 5/0402

(52) U.S. Cl. CPC A61B 5/055; A61B 5/0402
USPC 600/413; 600/410; 600/419; 600/420

(57) ABSTRACT

Selective excitation of spin magnetizations based on their velocities can be a useful tool for generating image contrast in magnetic resonance imaging (MRI) applications. Particularly in MR angiography, velocity-selective (VS) excitation can highlight arterial blood only by utilizing its significantly different velocity from stationary tissues and venous blood in the background. This invention describes the principle and design of MRI pulse sequences based on VS magnetization preparation. Its use for non-contrast enhanced MR angiography is demonstrated. The VS MRA compared to prior methods allows for large angiographic field-of-view and can generate positive angiographic contrast directly using single acquisition without subtraction.

### CROSS-REFERENCE TO RELATED APPLICATIONS

This application claims priority from U.S. Provisional Patent Application 61/704,619 filed Sep. 24, 2012, which is incorporated herein by reference.

### STATEMENT OF GOVERNMENT SPONSORED SUPPORT

This invention was made with Government support under grant no. 5R01 HL075803 awarded by the National Institutes of Health (NIH). The Government has certain rights in this invention.

### FIELD OF THE INVENTION

This invention relates to magnetic resonance angiography.

### BACKGROUND OF THE INVENTION

Magnetic resonance angiography (MRA) has evolved into a powerful diagnostic tool for arterial diseases due to its minimally invasive and nontoxic natures. The most established approach for MRA uses gadolinium-based contrast agents and T1 weighted imaging sequences to generate hyperintense arterial signals. Although the contrast-enhanced (CE) approach has shown excellent diagnostic performance in diverse applications, intravenous administration of contrast agents increases patient discomfort as well as examination costs and limits the achievable spatial resolution and artery-to-background contrast due to the requirement of short acquisition at the optimal post-injection time. Also, the risk of nephrogenic systemic fibrosis has not been completely cleared particularly in patients with renal failure.

Non-contrast-enhanced (NCE) MRA is free from the aforementioned limitations and has been an active MR research area in the past decade. The main technical goal of NCE MRA is to achieve high contrast between arteries and surrounding tissues that is comparable or superior to CE methods. Other important requirements include high spatial resolution in all three dimensions and large angiographic coverage. In addition to different tissue relaxation rates, the relatively fast movement of arterial blood has been a promising source of its contrast against background materials in NCE MRA.

Time-of-flight (TOF) imaging is the most established approach, and utilizes different exposure to RF excitation between stationary tissues within the imaging volume and inflowing fresh blood. However, the saturation effect of arterial blood that resides in the imaging slab limits artery-background contrast and possible slab thickness for 3D imaging. In another example, slice-selective (SS) saturation (or inversion)-prepared imaging can generate contrast between static materials within the saturated (or inverted) volume and arterial blood flowing into the imaging volume that did not experience the SS inversion. The main drawback is the loss of arterial blood that resides within the imaging volume which limits achievable artery-background contrast and angiographic coverage in the craniocaudal direction. Another recently introduced NCE MRA method employs flow-sensitive dephasing (FSD) preparation pulse that nulls arterial signal using intra-voxel dephasing effect of the FSD pulse. The resultant black artery image can be subtracted from a reference image that is acquired without the FSD preparation to generate a bright-artery angiogram. Although this approach enables large 3D angiographic coverage, the subtractive nature involves issues of doubling scan time and motion effects compared to single acquisition approaches.

With the present invention, we provide a new MRA method that overcomes the limitations of previous methods, allowing large 3D angiographic coverage and high spatial resolution in all three directions using a single acquisition without subtraction. The method of the present invention can be used with or without contrast agent administration. The feasibility of the method is demonstrated in NCE abdominal and peripheral MRA applications.

### SUMMARY OF THE INVENTION

The present invention provides a magnetic resonance imaging system and method for visualizing moving body tissue (e.g., arterial blood in MR angiography). A magnetic resonance imaging system is able to deliver a velocity-selective magnetization prepared imaging sequence to a body. This velocity-selective magnetization prepared imaging sequence includes a velocity-selective excitation pulse. This excitation pulse is played near or at a time of peak systolic arterial blood flow using cardiac triggering based on peripheral arterial pulsation or ECG signals. Following the delivery of the velocity-selective excitation pulse, imaging readouts are acquired by the magnetic resonance imaging system. The acquisition of the imaging data could employ balanced steady-state-free-precession (SSFP) readout or gradient-echo (GRE) readout or spin echo readout. In one embodiment, the method does not employ injecting a contrast agent, whereas in another embodiment the method does employ injecting a contrast agent such as gadopentetate dimeglumine (Gd-DTPA) and gadobenate dimglumine (Gd-BOPTA).

In one embodiment, the velocity-selective excitation pulse excites all spins by an excitation angle θ except arterial blood based on their velocities. In this case, the signal intensity of arterial blood is significantly higher than all other background tissues in the acquired MR image. The excitation angle θ is 90° for a velocity-selective saturation preparation, and the excitation angle θ is 180° for a velocity-selective inversion preparation.

In another embodiment, the velocity-selective magnetization pulse sequence excites all spins except arterial blood moving faster than a cut-off velocity. In this case, when the cut-off velocity is set to the maximum of normal arterial flow velocity, abnormally high arterial flow (e.g. flow jet due to stenosis or regurgitation) can be highlighted.

The velocity-selective excitation pulse in one example is defined according to a pulse sequence in a form of {A₁-G_{bp}-A₂- . . . -A_{N-1}-G_{bp}-A_{N}}, where G_{bp} is a bipolar gradient waveform, and Aᵢ is a complex value representing the amplitude and phase of i^{th} RF sub-pulse. {Aᵢ}_{i=1 to N} wherein N is the total number of RF sub-pulses, can be designed by a Shinnar-Le Roux algorithm or {Xᵢ}_{i=1 to N} can be designed by amplitude and frequency modulation functions for adiabatic full passage or a combination of adiabatic half passages.

The velocity-selective excitation pulse in another example is defined according to a pulse sequence in a form of {A₁-Gᵤₚ-T₁-180°-T₁-Gᵤₚ-A₂- . . . -A_{N-1}-Gᵤₚ-T_{N-1}-180°-T_{N-1}-Gᵤₚ-A_{N}}, where 180° represents an RF pulse for 180° spin rotation, Gᵤₚ is a unipolar gradient waveform, Aᵢ is a complex value representing the amplitude and phase of i^{th} RF sub-pulse, and Tᵢ is i^{th} delay time. The {Aᵢ}_{i=1 to N} can be designed by a Shinnar-Le Roux algorithm or adiabatic modulation functions. The {Tᵢ}_{i=1 to N-1} can be numerically optimized for the velocity-selective excitation pulse to be insensitive to field inhomogeneity and transmit RF inhomogeneity.

The MRA method according to embodiments of this invention based on velocity-selective excitation significantly relaxes the requirement of arterial inflow, and therefore allows large angiographic coverage and high spatial resolution in all three dimensions. The method generates positive angiographic contrast directly using single acquisition, and therefore reduces scan time and potential motion effects compared to all subtractive methods. Furthermore, relying on systolic arterial flow, the method is robust to arrhythmia which affects the diastolic period significantly but affects the systolic period only marginally.

### BRIEF DESCRIPTION OF THE DRAWINGS

FIG. 1 shows according to an exemplary embodiment of the invention a timing diagram of an MRA sequence using velocity-selective (VS) magnetization preparation. The pulse sequence is triggered by peripheral arterial pulsation or ECG signals, and has a VS magnetization preparation pulse, a delay time TI, a spectral-selective fat suppression pulse, and a segmented imaging readout. The VS preparation pulse tips down background tissues by θ, but does not substantially affect arterial blood moving in particular direction and speed. The VS pulse is played near or at the time of peak systolic flow by adjusting a trigger delay TD with respect to the cardiac trigger pulse, based on prior arterial flow measurements.

FIG. 2 shows according to an exemplary embodiment of the invention the principle of velocity-selective (VS) excitation under excitation k-space formalism. With kᵥ and k_{f} defined as reciprocal Fourier variables of velocity v and off-resonance f, respectively, RF subpulses played between a series of bipolar gradients deposit the B₁ field on kᵥ-k_{f} space in a discrete fashion. Each bipolar gradient yields an increment of kᵥ by Δkᵥ(=γ/2π∫₀^{T}^{_{bp}}(T_{bp}−T)G_{bp}(t)dt) and an increment of kᵥ by T_{bp} (=time duration of the bipolar gradint). The discrete B₁ deposit in the k-space causes excitation aliasing with a velocity FOV defined as the inverse of Δkᵥ. The ratio between the k_{f} and kᵥ increments (T_{bp}/Δkᵥ) determines the sensitivity to off-resonance which causes velocity profile shifting.

FIGS. 3A-C show an exemplary embodiment of a VS 180° inversion pulse sequence (FIG. 3A). Bloch simulation of the resultant longitudinal magnetization over the v-f plane (FIG. 3B) and for water on resonance (i.e., f=0) (FIG. 3C). The design parameters included velocity FOV=160 cm/s, velocity inversion bandwidth=60 cm/s, and a minimum-phase fitter design for the Shinnar-Le Roux transform. On resonance, the VS pulse inverts spins with a velocity range of [−10, +25] cm/s (stationary tissues and venous blood) while barely affecting spins with a velocity range of [−110, −30] cm/s (arterial blood) where a negative value corresponds to the direction of arterial flow

FIGS. 4A-D show an exemplary embodiment of a VS 90° saturation pulse sequence (FIG. 4A), Bloch simulation of the resultant longitudinal magnetization over the v-f plane (FIG. 4B), for off-resonance f=0, ±40 Hz (FIG. 4C), and for off-resonance f=0, ±80 Hz (FIG. 4D). The design parameters included velocity FOV=64 cm/s, velocity saturation bandwidth=17 cm/s, and a minimum-phase filter design for the Shinnar-Le Roux transform. A 90ₓ-180_{y}-90ₓ composite pulse train is inserted between every pairs of unipolar gradients to improve the sensitivity to off-resonance.

FIG. 5 shows an exemplary in-vivo result of peripheral VS-MRA. The refocused version of VS saturation preparation shown in FIGS. 4A-D and TI=0 are used. All major arteries are well visualized across a total of 90 cm-superior-inferior FOV (30 cm per station).

### DETAILED DESCRIPTION

An excitation pulse sequence with a desired spatial and velocity profile can be designed using the excitation k-space formalism under the small tip approximation. An excited transverse magnetization at position r with velocity v and off-resonance f can be represented by a Fourier transform of the radiofrequency (RE) B₁ field deposited in kᵣ-kᵥ-k_{f} space, where kᵣ, kᵥ, and k_{f} are reciprocal Fourier variables of r, v, and f, respectively.

$${M}_{xy} ( r , v , f ) = i γ {M}_{0} {\int }_{0}^{T} {B}_{1} ( t ) {e}^{i 2 π [ {k}_{r} \cdot r + {k}_{v} \cdot v + {k}_{f} f ]} d t \\ {k}_{r} ( t ) = \frac{γ}{2 π} {\int }_{t}^{T} G ( s ) d s , \\ {k}_{v} ( t ) = \frac{γ}{2 π} {\int }_{t}^{T} ( t - s ) G ( s ) d s , \\ {k}_{f} ( t ) = T - t \qquad [ 1 ]$$

where γ, M₀, and T are the gyromagnetic ratio, magnetization at equilibrium, and pulse duration, respectively. This is an extension of the conventional spatial-selective excitation by incorporating additional phase accrued by spin's velocity and off-resonance. The nominal velocity profile obtained by Eq. [1] can be shifted by vₒ along the velocity axis by modulating the phase of the Bᵢ field. That is, for a shifted velocity profile.

$${M}_{xy}^{′} ( r , v , f ) = {M}_{xy} ( r , v - {v}_{0} , f ) , \\ {B}_{1} ( t ) {e}^{j α ( t )} , \\ α ( t ) = γ {\int }_{t}^{T} ( t - s ) {v}_{0} \cdot G ( s ) d s \qquad [ 2 ]$$

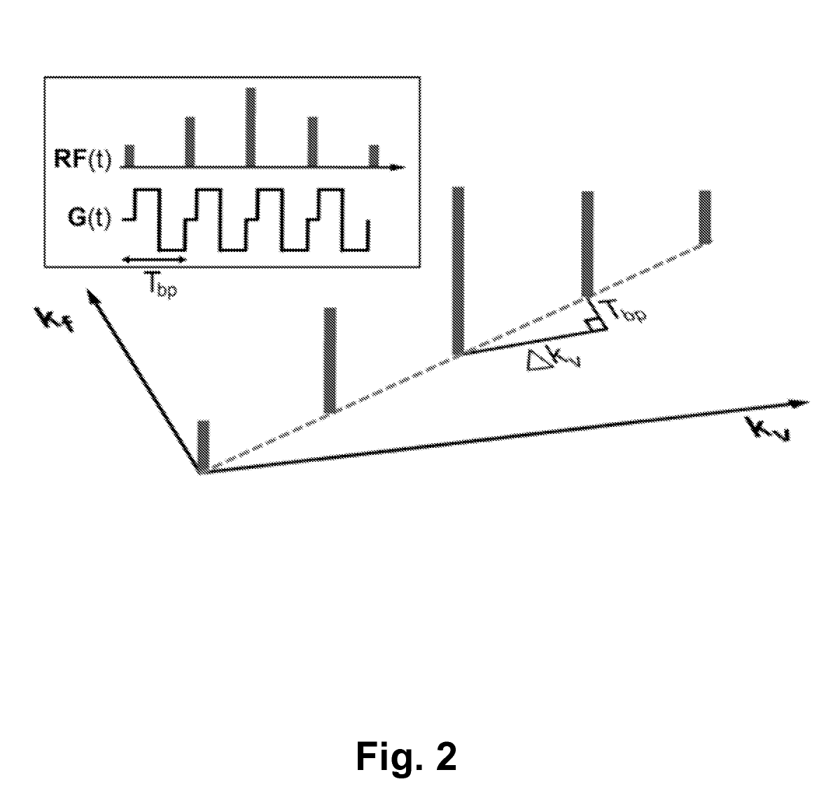

VS and non-spatial-selective excitation can be achieved by playing many (more than one) brief RE sub-pulses between a series of bipolar gradients as illustrated in FIG. 2. Each bipolar gradient G_{bp}(t) with a time duration of T_{bp} changes kᵥ by Δkᵥ=γ/2π∫₀^{T}^{_{bp}}(T_{bp}−t)G_{bp}(t)dt, changes k_{f} by T_{bp}, but does not change kᵣ due to the zero area of G_{bp}(1). In this way, the B₁ field is deposited only when kᵣ=0, which ensures no spatial selectivity, with a desired shape of the B₁ envelope that determines an excitation profile in the v-f plane. For large flip angles, the Fourier relation between the envelope of RF sub-pulses and the excitation profile would be less accurate. In this case, the RF envelope should be designed by the Shinnar-Le Roux transform that converts the RF design into a low-pass filter design problem. It should be noted that, due to the discrete deposition of the B₁ field, aliased excitation will occur at increments of Δkᵥ⁻¹ away from the desired excitation along the velocity axis, where Δkᵥ⁻¹ is termed as velocity FOV.

The effect of off-resonance can be explained by the excitation k-space trajectory tilted by the ratio of T_{bp}/Δkᵥ with respect to the k axis (dotted line in FIG. 2). Therefore, the excitation profile in the reciprocal v-f is also tilted by the same ratio, which manifest as velocity profile in proportion to off-resonance. The ratio T_{bp}/Δkᵥ monotonically increases as the velocity FOV increases, suggesting that a small velocity FOV is preferred to mitigate the off-ratio resonance effect. The quadratic relationship between the ratio and the velocity FOV can be shown analytically as well. Δkᵥ is proportional to the first moment of gradient and thus proportional to T_{bp}². Therefore, the ratio T_{bp}/Δkᵥ is proportional to T_{bp}⁻¹, and the velocity FOV (=Δkᵥ⁻¹) is proportional to T_{bp}⁻².

Methods

Pulse Sequence

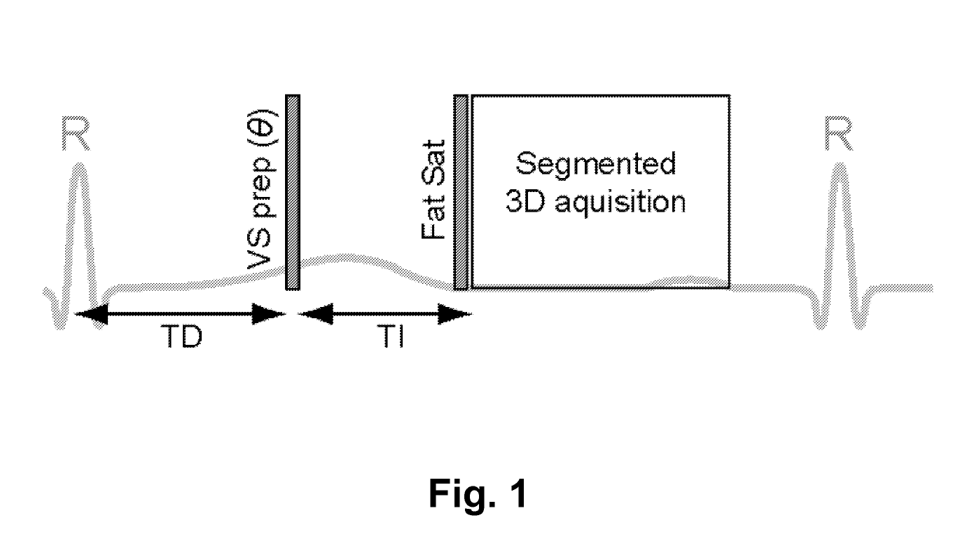

The pulse sequence for the VS MRA is triggered by peripheral arterial pulsation or ECG signals. The sequence includes a VS excitation pulse with flip angle of θ after a cardiac trigger delay (TD), a zero or positive delay (TI), a spectrally-selective fat suppression pulse, and a segmented imaging readout (FIG. 1). The TD is determined such that the VS preparation pulse is played near or at the time of peak arterial flow to ensure large velocity difference between arterial blood and background tissues.

VS Excitation Pulse

A VS excitation pulse sequence involves several design parameters to be adjusted. Ideally, the velocity pass-band should have the largest possible upper bound, the smallest possible lower bound, and the narrowest possible transition-band to include various types of arterial flow. The achievable upper bound is limited by the preference to a small velocity FOV for reducing off-resonance-induced profile shifting, and the transition sharpness is traded off by a long pulse duration (or large number of RF sub-pulses). The stop-bandwidth should be minimized to increase the upper bound of the pass-band for a given velocity FOV. In designing a VS pulse, therefore, we sought the smallest possible velocity FOV and the largest possible number of RF sub-pulses that allow most of arterial blood and venous blood to be included in the pass-band and inversion-band, while limiting the pulse duration to less than 20 ms.

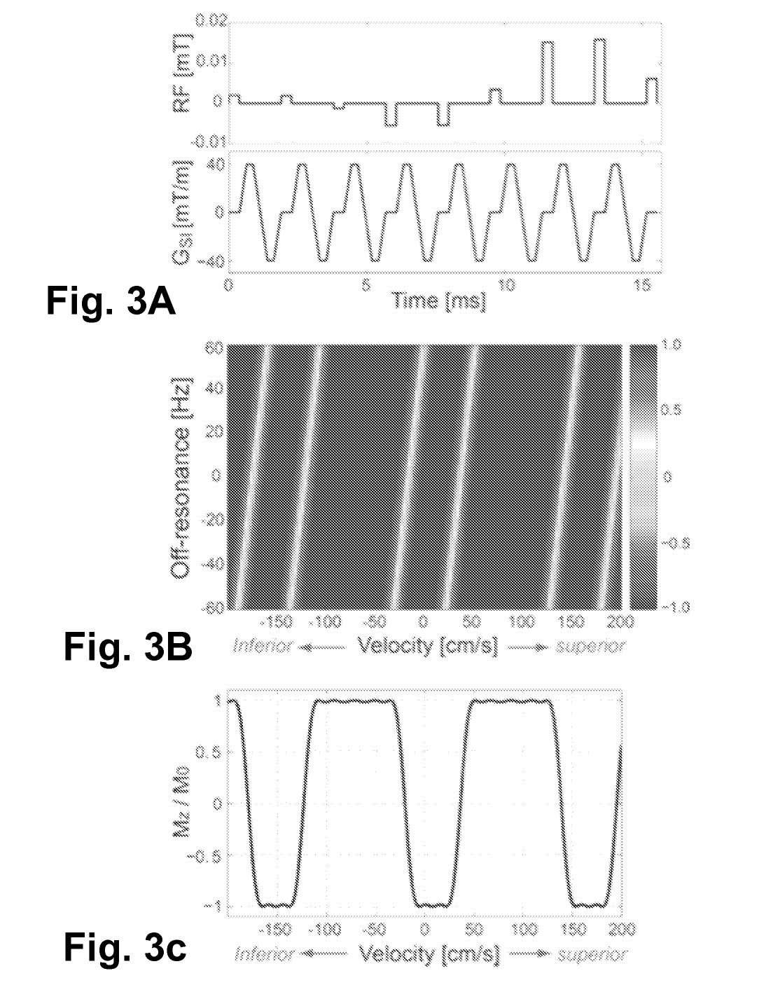

FIG. 3A shows the VS 180° inversion pulse sequence used in examples of this invention, FIG. 3B shows a Bloch simulation of the resultant longitudinal magnetization over v-f plane, and FIG. 3C shows for water on resonance (i.e., f=0). In FIGS. 3B and 3C, a positive velocity indicates flow moving superiorly (i.e., the direction of venous flow). The design parameters included velocity FOV=160 cm/s, velocity inversion bandwidth=60 cm/s (fun-width-half-maximum), and the number of RF sub-pulses=9. A bipolar gradient waveform was designed to generate Δkᵥ of 1/160 s/cm (the inverse of the velocity FOV) using a maximum gradient amplitude of 40 mT/m and a maximum gradient slew rate of 150 T/m/s, and was 1.5 ms long. The velocity profile shifting by unit off-resonance (i.e., the ratio T_{bp}/Δkᵥ) was 0.24 cm/s/Hz. A minimum-phase filter was used for the Shinnar-Le Roux transform in designing the envelope of RF sub-pulses to reduce the pulse duration without compromising inversion performance. The nominal velocity profile, initially centered at 0 cm/s, was shifted by 8 cm/s in the superior direction using the RE phase modulation (Eq. [2]).

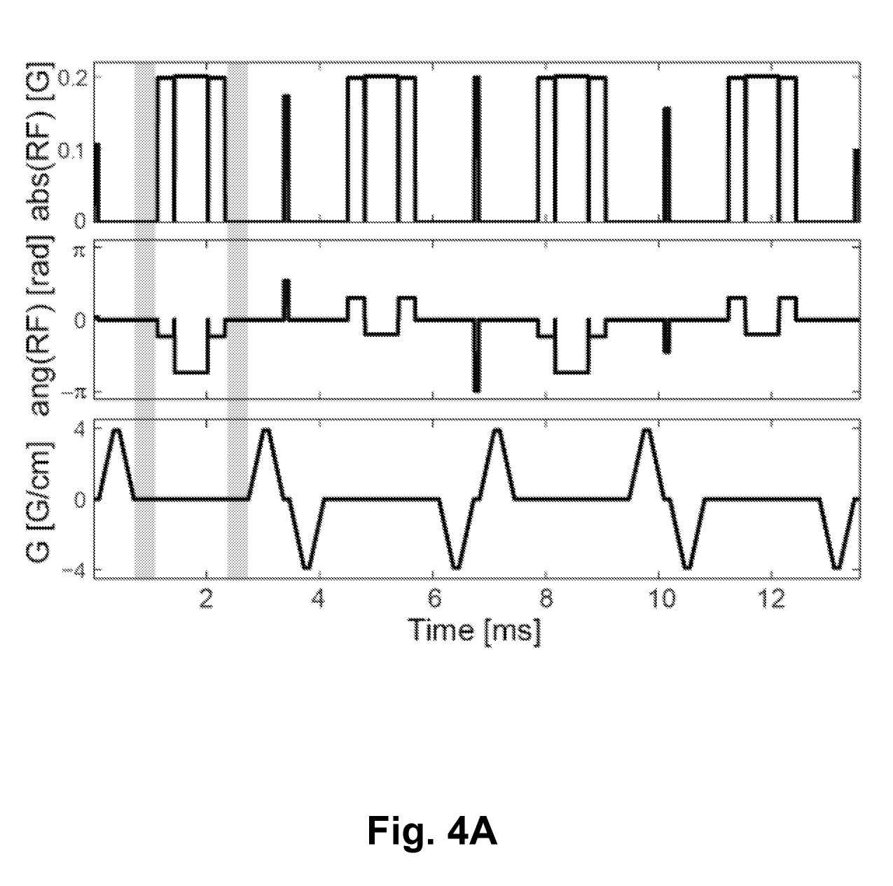

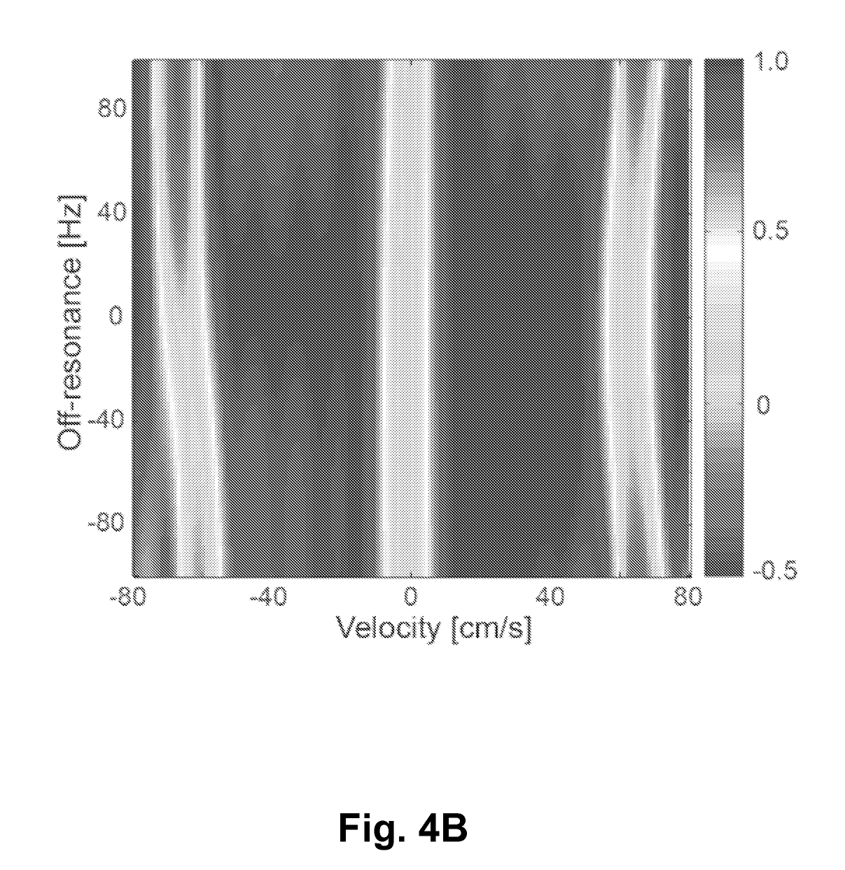

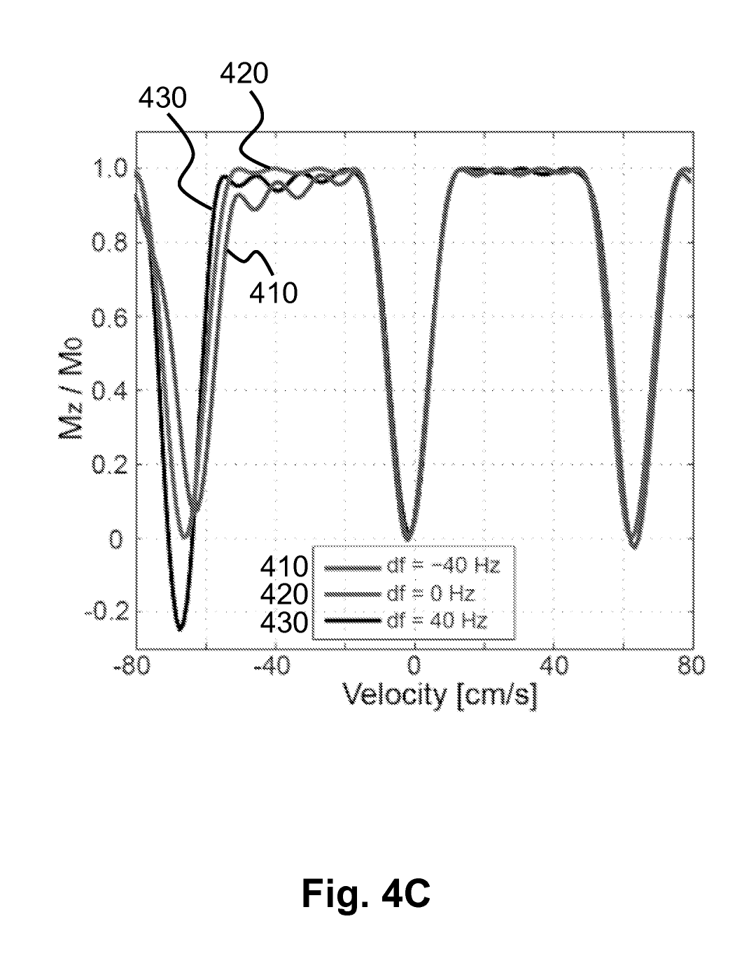

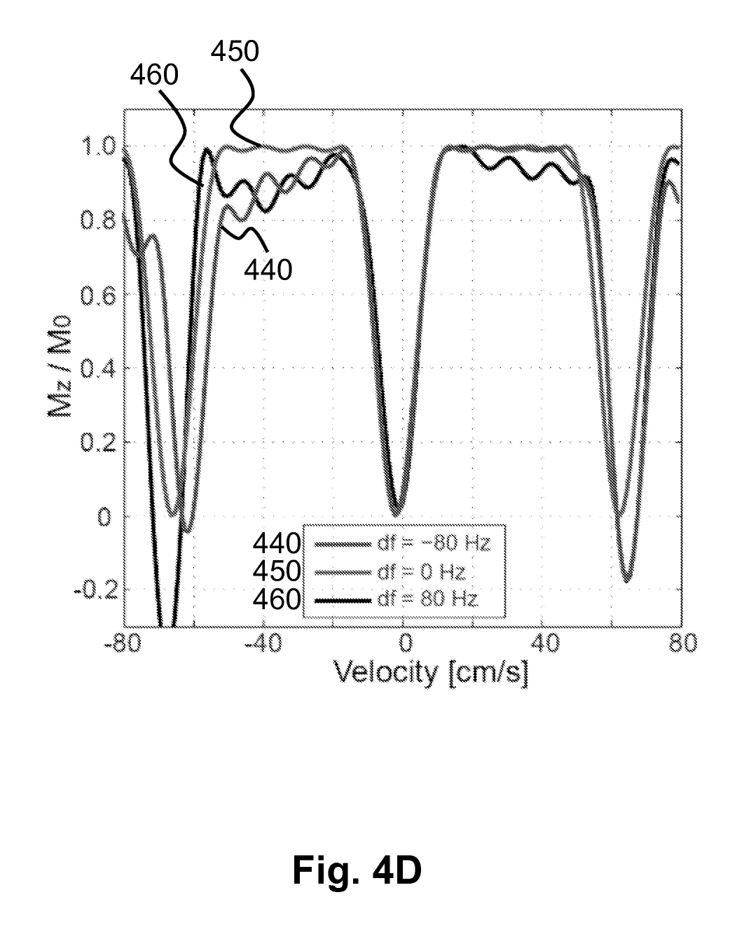

Another possible design of a VS excitation pulse incorporates 180° refocusing pulses between the halves of bipolar gradients. With this so-called refocused design, the phase accrued by off-resonance during the period of the first unipolar will be cancelled out by the phase accrued in the opposite direction during the period of the second unipolar, which significantly reduces the off-resonance sensitivity. FIGS. 4A-D show a VS 90° saturation pulse sequence designed by incorporating refocusing pulses, and a Bloch simulation of the resultant longitudinal magnetization. The design parameters include velocity FOV=64 cm/s, full-width-half-maximum excitation bandwidth=17 cm/s, velocity shifting=−2 cm/s. A 1.2-ms-long composite pulse train of 90ₓ-180_{y}-90ₓ is used for B₁-robust 180° rotation. The simulation results show that the effect of off-resonance on the longitudinal magnetization Mz profile is significantly reduced.

Imaging Protocol

In vivo experiments were performed on a 1.5T clinical whole-body MR system (Signa HDx; GE Healthcare, Waukesha, Wis.). The body coil was used for RF excitation. An eight-channel cardiac-array coil was used for signal reception.

NCE MRA scans were performed on human subjects using two protocols that target (i) abdominopelvic arteries and (ii) peripheral arteries.
i. Abdominal MRA was performed using the VS inversion preparation described in FIG. 3 on six healthy subjects. Imaging parameters included imaging orientation=coronal, TI=700 ms, flip angle=70°, spatial resolution=1.4×1.4×2.0 mm³, FOV=340×300×120 mm³, TR=4.7 ms, readout bandwidth=125 kHz, 2-fold acceleration using iterative self-consistent parallel imaging reconstruction with 32 self-calibration lines, number of phase encodes per acquisition block=61, acquisition time per respiratory cycle=287 ms. Conventional slice-selective inversion recovery (SS-IR) imaging was performed for using the same imaging parameters. The VS inversion pulse was played at the time of peak systolic flow measured at the isocenter, whereas the SS inversion pulse was played at the time of the onset of systolic flow measured at the 120-mm superior position.
ii. Peripheral MRA was performed using the refocused VS saturation preparation described in FIG. 4 on healthy subjects. Imaging parameters were imaging orientation=coronal, TI=0 ms, flip angle α=70°, spatial resolution=1.1×1.1×1.3 mm³, FOV=30×32×9.1 cm³, number of coronal slices=70, TR=4.5 ms, readout bandwidth=125 kHz, number of phase encodes per acquisition block=73, acquisition time per acquisition block=329 ms, and scan time=256 heart beats (3.7 min based on 70 beats/min).

Results

Representative coronal MIP images of abdominal VS MRA in three subjects are shown in Appendix A in the provisional application to which this application claims the benefit (referred to as FIG. 6 in Appendix A). The abdominal aorta, renal arteries, and iliac arteries are well visualized, which demonstrates successful VS separation of arterial blood from background tissues over the entire superior-inferior (SI) FOV. With a TI of 700 ms, background signal is well suppressed except in the intestine that contains short TI contents.

A comparison between abdominal VS and SS MRA in the same subject is shown in Appendix A in the provisional application to which this application claims the benefit (referred to as FIG. 7 in Appendix A). Although VS MRA with TI=700 ms yields excellent artery visualization, SS MRA with the same TI is only able to visualize the abdominal aorta up to approximately 100 mm from the top of the FOV due to limited arterial inflow. The extent of the aorta is significantly increased with TI=1200 ms, but only up to the beginning of iliac arteries. Furthermore, due to the longer TI, background signal is increased compared to the case of TI=700 ms.

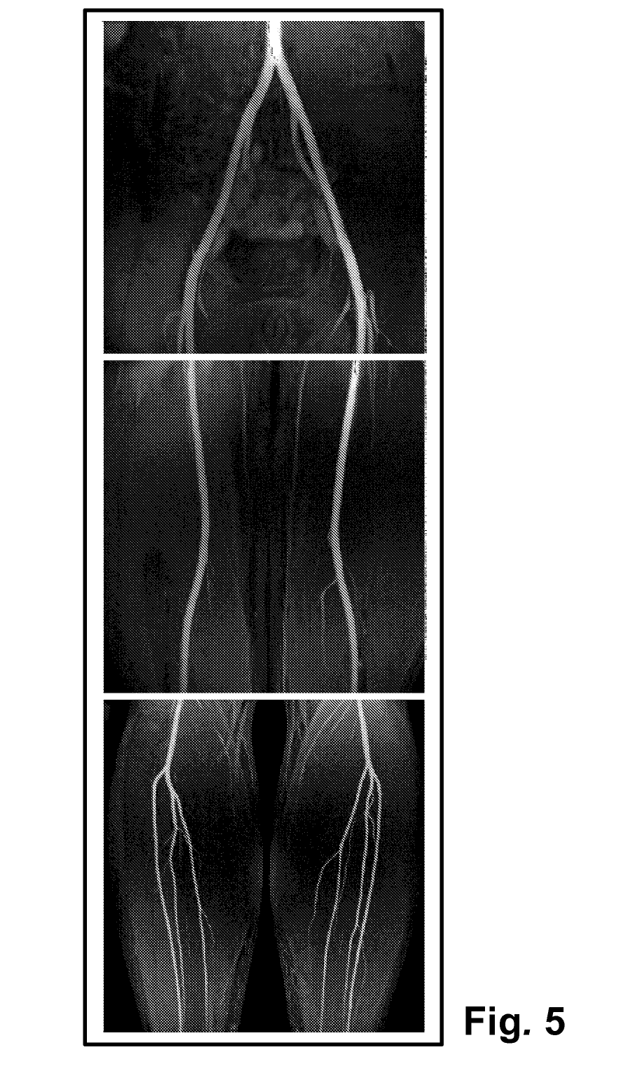

FIG. 5 shows peripheral angiograms obtained using the refocused VS MRA method in three stations for the pelvis, thighs and calves. Iliac, femoral, popliteal, and tibial arteries are clearly delineated across a total of 90 cm-S/I FOV (30 cm per station). The angiograms show excellent suppression of the vein, muscle, and synovial fluid in all three stations.

Variations

The VS excitation flip angle and the subsequent delay time TI determine the tradeoff between inflow effects and background suppression. With 180° flip angle, for instance, we can use long TI and therefore increase arterial inflow effects. However, background suppression will be sub-optimal whenever there are multiple T1 species. In another example, the combination of 90° flip angle and zero delay can achieve T1 independent background suppression but allows no inflow time. Other combinations of VS flip angles (between 90° and 180°) and TIs (>0) will yield intermediate effects between these two cases.

The envelope of RF subpulses determines the shape of excitation profile along the velocity axis, and can be designed in different ways. One way is to use the inversion Fourier transform of a desired excitation profile over velocity. The Shinnar-Le Roux algorithm is more accurate and flexible in designing the RF envelope function particularly for large excitation flip angles. Another possibility is to use adiabatic full passage or a combination of adiabatic half passage functions, which is be robust to transmit RF inhomogeneity.

The VS excitation pulse sequence can be extended to acceleration-selective excitation by replacing the bipolar gradient waveform with a tripolar gradient waveform and depositing the B1 field along kₐ (=Fourier variable of acceleration) in the excitation k-space. Acceleration-selective excitation may outperform the VS excitation when arterial blood has a small velocity, but a relatively large acceleration during the systolic period.

What is claimed is:

1. A magnetic resonance imaging method for visualizing moving body tissue, comprising:
a magnetic resonance imaging system with a velocity-selective magnetization-prepared imaging sequence, wherein said velocity-selective magnetization-prepared imaging sequence comprises of a velocity-selective excitation pulse; and
acquiring imaging readouts from said magnetic resonance imaging system.

2. The method as set for in claim 1, wherein said velocity-selective magnetization prepared imaging sequence is triggered by physiological signals, wherein said physiological signals contain peripheral arterial pulsation or ECG signals.

3. The method as set forth in claim 1, wherein said acquiring imaging readouts is preceded by material-specific excitation pulses, wherein said material-specific excitation pulses contain a fat suppression pulse, a T1-weighted magnetization preparation pulse or a T2-weighted magnetization preparation pulse.

4. The method as set forth in claim 1, wherein said velocity-selective excitation pulse is played near or at a time of peak systolic arterial flow.

5. The method as set forth in claim 1, wherein said velocity-selective excitation pulse excites all spins by an excitation angle θ except arterial blood based on their velocities, and wherein said acquiring of said imaging readouts occurs with a zero or positive delay time after the application of said velocity-selective excitation pulse.

6. The method as set forth in claim 5, wherein said excitation angle θ is 90° for a velocity-selective saturation preparation.

7. The method as set forth in claim 5, wherein said excitation angle θ is 180° for a velocity-selective inversion preparation.

8. The method as set forth in claim 1, wherein said velocity-selective excitation pulse excites all spins except arterial blood moving faster than a cut-off velocity, and wherein said acquiring of said imaging readouts occurs with a zero or positive delay time after the application of said velocity-selective excitation pulse.

9. The method as set forth in claim 1, wherein said velocity-selective excitation pulse is according to a pulse sequence in a form of {A₁-G_{bp}-A₂- . . . -A_{N-1}-G_{bp}-A_{N}}, wherein said G_{bp} is a bipolar gradient waveform, and said Aᵢ is a complex value representing the amplitude and phase of i^{th} RF sub-pulse, and said N is the total number of RF sub-pulses.

10. The method as set forth in claim 9, wherein said {Aᵢ}_{i=1 to N} is designed by a Shinnar-Le Roux algorithm.

11. The method as set forth in claim 9, wherein said {Aᵢ}_{i=1 to N} is designed by amplitude and frequency modulation functions for adiabatic full passage or a combination of adiabatic half passages.

12. The method as set forth in claim 1, wherein said velocity-selective excitation pulse is according to a pulse sequence in a form of {A₁-Gᵤₚ-T₁-180°-T₁-Gᵤₚ-A₂- . . . -A_{N-1}-Gᵤₚ-T_{N-1}-180°-T_{N-1}-Gᵤₚ-A_{N}}, wherein the 180° represents RF pulses for 180° spin rotation, Gᵤₚ is a unipolar gradient waveform, Aᵢ is a complex value representing the amplitude and phase of i^{th} RF sub-pulse, and Tᵢ is i^{th} delay time.

13. The method as set forth in claim 12, wherein said {Aᵢ}_{i=1 to N} is designed by a Shinnar-Le Roux algorithm.

14. The method as set forth in claim 12, wherein said {Aᵢ}_{i=1 to N} is designed by amplitude and frequency modulation functions for adiabatic full passage or a combination of adiabatic half passages.

15. The method as set forth in claim 12, wherein said the {Tᵢ}_{i=1 to N-1} is numerically optimized for said velocity-selective excitation pulse to be insensitive to field inhomogeneity and transmit RF inhomogeneity.

16. The method as set forth in claim 12, wherein said 180° spin rotation is implemented by composite pulse trains in a form of {Y_{1,β}^{¹}-Y_{2,β}_{₂}- . . . -Y_{M,β}_{_{M}}} wherein said Yᵢ represent the flip angle of i^{th} RF pulse, and wherein said βᵢ represent the angle of the rotation axis of the i^{th} RF pulse, and wherein said M is the total number of RF pulses.

17. The method as set forth in claim 12, wherein said 180° spin rotation is implemented by adiabatic full passage or adiabatic half passage pulses.

18. The method as set forth in claim 1, wherein said acquiring said imaging data employs a balanced steady-state-free-precession (SSFP) readout, a gradient-echo (GRE) readout or a spin-echo readout.

19. The method as set forth in claim 1, wherein said acquiring said imaging readouts comprises employing parallel imaging, wherein said parallel imaging comprises a generalized auto-calibrating partially parallel acquisition (GRAPPA), a self-consistent parallel imaging reconstruction (SPIRiT) or a sensitivity encoding (SENSE).

20. The method as set forth in claim 1, wherein said method (i) does not employ injecting a contrast agent or (ii) does employ injecting a contrast agent.
